(19) United States
(12) Patent Application Publication
(10) Pub. No.: US 2021/0292438 A1
(43) Pub. Date: Sep. 23, 2021

(54) CONDITIONAL INTERNALIZATION OF PEGYLATED AGENTS BY PRETARGETING BI-SPECIFIC PEG-BINDING ANTIBODIES FOR DIAGNOSIS AND THERAPY

(71) Applicant: ACADEMIA SINICA, Taipei City (TW)

(72) Inventors: Steve ROFFLER, Taipei City (TW); Tian-Lu Cheng, Kaohsiung City (TW); Yu-Cheng Su, Taipei City (TW); Kuo-Hsiang Chuang, Taipei City (TW)

(73) Assignee: ACADEMIA SINICA, Taipei City (TW)

(21) Appl. No.: 17/340,102

(22) Filed: Jun. 7, 2021

Related U.S. Application Data
(62) Division of application No. 16/615,822, filed on Nov. 22, 2019.
(60) Provisional application No. 62/510,046, filed on May 23, 2017.

(51) Int. Cl. C07K 16/44; A61K 47/60; A61K 47/69; A61P 35/00; A61K 31/55; A61K 31/704; A61K 51/06; C07K 16/28; C07K 16/30; C07K 16/32; G01N 33/574

(52) U.S. Cl. CPC C07K 16/44; A61K 47/60; A61K 47/6915; A61P 35/00; A61K 31/55; A61K 2039/505; A61K 51/065; C07K 16/2863; C07K 16/30; C07K 16/32; G01N 33/57492; A61K 31/704

(57) ABSTRACT

A monomeric bispecific polyethylene glycol (PEG) engager that includes an anti-PEG Fab fused to a disulfide stabilized scFv that specifically binds to a cell surface antigen. The PEG engager, in the absence of PEG, remains monomeric upon binding to the cell surface antigen on a cell and remains on the surface of the cell. Also disclosed is a method for treating cancer by administering a PEG engager followed by a PEGylated anti-cancer agent. Further disclosed is a method for diagnosing a cell-mediated disorder by administering a PEG engager followed by a PEGylated diagnostic agent.

This patent application is a Divisional application of U.S. patent application Ser. No. 16/615,822, filed Nov. 22, 2019, which claims the benefit of U.S. National Stage Application of PCT/US2018/031784 filed on May 9, 2018, which claims the benefit of U.S. Patent Application No. 62/510,046, filed May 23, 2017, the content of each of which is incorporated herein by reference in their entirety. This application contains a Sequence listing in computer readable form, which is also incorporated herein by reference in its entirety.

### DESCRIPTION OF PRIOR ART

Triple-negative breast cancer (TNBC) represents 11.2-16.3% of all breast cancers. TNBC cells do not express estrogen receptors, progesterone receptors, and human epidermal growth factor receptor 2. TNBC, typically aggressive, is associated with a poor prognosis. Limited treatment options are available due to the absence of well-defined therapeutic targets.

Systemic chemotherapy has been the primary therapeutic option for TNBC until it was discovered that epidermal growth factor receptor (EGFR) is overexpressed in 50% of TNBC tumors. EGFR-targeted agents, such as tyrosine kinase inhibitors, are under development for the treatment of TNBC. However, EGFR-targeted tyrosine kinase inhibitors such as gefitinib and erlotinib show minimal effectiveness in TNBC patients.

Nanomedicines, i.e., nanosized drug-containing particles, are an attractive alternative to systemic chemotherapy. Nanomedicines favorably alter the pharmacokinetic profile of chemotherapy drugs, reduce off-target toxicity, and improve the therapeutic index. Nanomedicines passively accumulate in tumors as a result of enhanced permeability and retention effect in the tumor environment where leaky blood vasculature combines with impaired lymphatic drainage. Lung, breast, and ovarian tumors all display high accumulation of nanosized particles.

Nanomedicines such as polyethylene glycol modified, i.e., PEGylated, liposomal doxorubicin are currently being investigated for the treatment of TNBC. PEG is employed to increase half-life by decreasing recognition and clearance by the reticuloendothelial system, i.e., the so-called “stealth” feature of PEGylation.

The effectiveness of nanomedicines can be improved via active targeting by functionalizing the surface of nanocarriers with targeting ligands that bind to endocytic receptors on cancer cells. Such targeting promotes receptor-mediated endocytosis, resulting in increased cellular uptake of nanomedicines with concomitant improved anti-tumor activity. Yet, many technical hurdles must be overcome to produce new more effective nanocarriers. For example, attachment of targeting ligands can compromise the stealth feature of PEGylated nanocarriers and hinder their uptake into a tumor.

The need exists to develop cancer therapeutics that are both more effective against cancer cells while having fewer off-target side effects. The microfilter structure may be a solid membrane structured with microholes to make it a semi-permeable medium. The microfilter structure has been commonly applied to separate micro objects from complex samples.

### SUMMARY

To meet the need set forth above, a monomeric bispecific PEG engager is provided. It contains an anti-PEG Fab fused to a disulfide stabilized scFv that specifically binds to a cell surface target. In the absence of PEG, the PEG engager remains monomeric upon binding to the cell surface target on a cell and remains on the surface of the cell.

Also disclosed is a method for treating cancer. The treatment method includes (i) identifying a subject suffering from cancer, (ii) administering to the subject a monomeric bispecific PEG engager that specifically binds to PEG and to a target on cancer cells in the subject, and (iii) subsequently administering to the subject a PEGylated anti-cancer agent. The anti-cancer agent is internalized into the cancer cells upon binding to the monomeric bispecific PEG engager bound to the cancer cells, thereby killing the cancer cells.

Further provided is a kit for treating an epidermal growth factor expressing (EGFR-positive) cancer, the kit containing a monomeric bispecific PEG engager that specifically binds to PEG and to an EGF receptor, and PEGylated anti-cancer agent.

Another kit within the scope of the invention is for diagnosing an EGFR-positive cancer. The kit includes a monomeric bispecific PEG engager that specifically binds to PEG and to an EGF receptor, and a PEGylated imaging agent.

Moreover, a method for cell imaging is provided that includes the steps of (i) contacting a cell with a monomeric bispecific PEG engager that specifically binds to PEG and to a target on the cell, (ii) subsequently contacting the cell with a PEGylated imaging agent, and (iii) detecting the presence of the PEGylated imaging agent. The PEGylated imaging agent is internalized into the cell upon binding to the monomeric bispecific PEG engager bound to the cell.

A method for diagnosing a cell-mediated disorder is also disclosed. The method is carried out by administering to a subject a monomeric bispecific PEG engager that specifically binds to PEG and to a target on cells mediating the disorder, subsequently administering to the subject a PEGylated diagnostic agent, and detecting the location of the PEGylated diagnostic agent. The subject is diagnosed as suffering from the cell-mediated disorder, e.g., cancer, if the PEGylated diagnostic agent is located in the cells upon binding to the monomeric bispecific PEG engager bound to the cells.

The details of one or more embodiments of the invention are set forth in the description and drawings below. Other features, objects, and advantages of the invention will be apparent from the description, from the drawings, and from the appended claims. All references cited herein are hereby incorporated by reference in their entireties.

### BRIEF DESCRIPTION OF THE DRAWINGS

The invention description below refers to the accompanying drawings, of which:

FIG. 1A is a bar graph showing percentage of PEG engager^{EGFR} internalized into cells treated with (open bars) or without (filled bars) PEG-quantum dot 655 (PEG-Qdot655) at different times quantified from confocal images of individual cells (n=15). Representative confocal images from two independent experiments are shown. Data is mean±s.d. **P≤0.001, ***P≤0.0001 (two-way analysis of variance), n.s.=not significant.

FIG. 1B is a bar graph showing percentage of PEG engager^{EGFR} co-localized with lysosomes stained with LysoTracker Red DND-99 at the indicated times quantified from confocal images of individual cells (n=15). Significance values are as shown in the legend to FIG. 1A above.

FIG. 2A is a plot of cell proliferation as a percent of control versus doxorubicin concentration for BT-20 cells treated as indicated in the legend. The data is representative of three independent experiments.

FIG. 2B is a plot of cell proliferation as a percent of control versus doxorubicin concentration for MDA-MB-468 cells treated as indicated in the legend shown in FIG. 2A.

FIG. 2C is a plot of cell proliferation as a percent of control versus doxorubicin concentration for MDA-MB-231 cells treated as indicated in the legend shown in FIG. 2A.

FIG. 2D is a bar graph showing the half maximal effective concentration (EC₅₀) of PEG engager^{EGFR} plus Doxisome and PEG engager^{CD19} plus Doxisome for inhibiting proliferation of BT-20, MDA-MB-468 and MDA-MB-231 cells. Data is shown as mean±s.d. Significant differences in mean EC₅₀ values are indicated as follows: **P≤0.001, ***P≤0.0001 (two-way analysis of variance).

FIG. 3A is a plot of mean tumor size±standard deviation versus days post-treatment of SCID mice bearing an MDA-MB-468 tumor (n=8). The treatment modalities, administered on the days indicated by arrows, are shown below the plot.

FIG. 3B is a plot of mean body weights±standard deviation versus time of MDA-MB-468 mice treated as indicated below FIG. 3A on the marked days (n=8). LD=liposomal doxorubicin, i.e., Doxisome.

FIG. 3C shows mean±standard deviation of tumor sizes in groups of 6 SCID mice 43 days after being treated as indicated below FIG. 3A once per week for 4 weeks. Statistical analysis of the differences in tumor volumes between treatment and control groups was performed by one-way analysis of variance (ANOVA) followed by Dunnett's multiple comparisons. *p≤0.05, **p≤0.005.

### DESCRIPTION OF THE INVENTION

As mentioned above, a monomeric bispecific polyethylene glycol engager (PEG engager) is disclosed that includes an anti-PEG Fab fused to a disulfide stabilized scFv.

The anti-PEG Fab binds specifically to PEG. In a specific example, the Fab includes a heavy-chain CDR1 having the sequence of SEQ ID NO: 3, a heavy-chain CDR2 having the sequence of SEQ ID NO: 4, a heavy-chain CDR3 having the sequence of SEQ ID NO: 5, a light-chain CDR1 having the sequence of SEQ ID NO: 6, a light-chain CDR2 having the sequence of SEQ ID NO: 7, and a light-chain CDR3 having the sequence of SEQ ID NO: 8.

The disulfide stabilized scFv mentioned above specifically binds to a cell surface antigen. The cell surface antigen is expressed on the surface of a target cell, e.g., a cancer cell. The cell surface antigen can be a protein, a carbohydrate, or a lipid. For example, the cell surface antigen can be a growth factor receptor. The growth factor receptor can be, but is not limited to the epidermal growth factor receptor (EGFR), an insulin-like growth factor receptor, human epidermal growth factor receptor 2 (HER2), HER3, HER4, and c-Met. In a specific example, the cell surface protein is EGFR.

Additional examples of cell surface antigens include CD19, CD20, CD5, CD21, CD25, CD37, CD30, CD33, CD45, CAMPATH-1, A33, G250, folate-binding protein, PSMA, GD2, GD3, GM2, Lewis Y, CA-125, CA19-9, IL2 receptor, tenascin, metalloproteinases, and FAP.

In the absence of PEG, the PEG engager remains monomeric upon binding to the cell surface antigen on a cell. For example, if the PEG engager includes a disulfide stabilized scFv that specifically binds to EGFR, the PEG engager binds to the EGFR without activating this receptor and initiating internalization, thereby remaining bound on the cell surface.

In this connection, the PEG-engager can include a fluorescent label, e.g., Alexa Fluor 647, for labeling a cell surface.

The monomeric bispecific PEG engager can be used in a method for treating cancer. The cancer can be any cancer that is characterized by overexpression of EGFR including, but not limited to, breast cancer, lung cancer, ovarian cancer, head and neck cancer, colon cancer, kidney cancer, prostate cancer, liver cancer, and cervical cancer. In a specific example, the cancer is TNBC.

The cancer treatment method is accomplished by administering at least two agents to a cancer patient sequentially as follows.

The first agent administered is the monomeric bispecific PEG engager described above that specifically binds to PEG and to a target on cancer cells in the patient. The target can be a growth factor receptor selected from EGFR, an insulin-like growth factor receptor, HER2, HER3, HER4, or c-Met. In an exemplary method, the target is EGFR.

The monomeric bispecific PEG engager, in the absence of PEG, remains monomeric upon binding to the target on cancer cells and stays bound to the cell surface until internalization is initiated by binding to PEG.

The second agent administered is a PEGylated anti-cancer agent, e.g., PEGylated liposomal doxorubicin or PEGylated liposomal vinorelbine. The PEGylated anti-cancer agent is internalized into the cancer cells upon binding to the monomeric bispecific PEG engager bound to the cancer cells, thereby killing the cancer cells.

An exemplary method for treating TNBC is carried out by first administering a monomeric PEG engager that specifically binds to EGFR followed by administering PEGylated liposomal doxorubicin.

Certain cancerous tumors are characterized by heterogeneity of cancer cells within the tumor. The cancer treatment method described above can be adapted for treating such tumors by administering two distinct PEG engagers, each specifically binding to a distinct target on cancer cells. Both PEG engagers bind to PEG, yet can bind to different subsets of cancer cells in a tumor. The PEGylated anti-cancer agent mentioned above. is also administered after administering both PEG engagers.

In a particular example of this method, a PEG engager that specifically binds to EGFR is administered together with a second PEG engager that specifically binds to an insulin-like growth factor receptor, HER2, HER3, HER4, or c-Met, followed by administering PEGylated liposomal doxorubicin or PEGylated liposomal vinorelbine.

As mentioned above, a kit is provided for treating an EGFR-positive cancer via the above method. An exemplary kit for treating triple-negative breast cancer contains a monomeric bispecific PEG engager that specifically binds to PEG and to a growth factor receptor selected from EGFR, an insulin-like growth factor receptor, HER2, HER3, HER4, or c-Met. The kit also contains a PEGylated anti-cancer agent.

A specific kit contains (i) a monomeric bispecific PEG engager that specifically binds to PEG and to EGFR and (ii) PEGylated liposomal doxorubicin. The monomeric bispecific PEG engager can contain an Fab fragment including a heavy-chain CDR1 having the sequence of SEQ ID NO: 3, a heavy-chain CDR2 having the sequence of SEQ ID NO: 4, a heavy-chain CDR3 having the sequence of SEQ ID NO: 5, a light-chain CDR1 having the sequence of SEQ ID NO: 6, a light-chain CDR2 having the sequence of SEQ ID NO: 7, and a light-chain CDR3 having the sequence of SEQ ID NO: 8.

Another kit provided is for diagnosing an EGFR-positive cancer. The kit includes a monomeric bispecific PEG engager that specifically binds to PEG and to an EGF receptor, and a PEGylated imaging agent, e.g., a fluorescently or radioactively labeled PEGylated nanoparticle. The monomeric bispecific PEG engager can be that described in the preceding paragraph.

Also mentioned, supra, is a cell imaging method. The method can be carried out using any of the monomeric bispecific PEG engagers set forth above. The imaging is accomplished by detecting the presence of a PEGylated imaging agent, which can be, but is not limited to, a fluorescently or radioactively labeled PEGylated nanoparticle.

Another method for diagnosing a cell-mediated disorder is discussed above. The method is carried out by administering to a subject a monomeric bispecific PEG engager that specifically binds to PEG and to a target on cells mediating the disorder. Like the method described in the preceding paragraph, this method can employ one or more of the monomeric bispecific PEG engagers set forth, supra. The PEGylated diagnostic agent can be, e.g., a fluorescently or radioactively labeled PEGylated nanoparticle.

Without further elaboration, it is believed that one skilled in the art can, based on the description above, utilize the present invention to its fullest extent. The specific examples below are to be construed as merely illustrative, and not limitative of the remainder of the disclosure in any way whatsoever.

The present invention further provides a method for treating cancer, comprising: (1) identifying a subject suffering from cancer; (2) administering to the subject at least one monomeric bispecific polyethylene glycol (PEG) engager that specifically binds to PEG and to targets on cancer cells in the subject; and (3) administering to the subject a PEGylated anti-cancer agent after receiving the at least one monomeric bispecific PEG engager, wherein the PEGylated anti-cancer agent is internalized into the cancer cells upon binding to the monomeric bispecific PEG engager bound to the cancer cells, thereby killing the cancer cells.

In one embodiment, the PEGylated anti-cancer agent is administered after the clearance rate of the at least one monomeric bispecific PEG engager is higher than 90% in the subject.

In another embodiment, the targets comprise epidermal growth factor receptor (EGFR), insulin-like growth factor receptor, human epidermal growth factor receptor 2 (HER2), HER3, HER4, c-Met, CD19, CD20, CD5, CD21, CD25, CD37, CD30, CD33, CD45, CAMPATH-1, A33, G250, folate-binding protein, PSMA, GD2, GD3, GM2, Lewis Y, CA-125, CA19-9, IL2 receptor, tenascin, metalloproteinases or FAP. In a preferred embodiment, the targets comprise EGFR, CD19 or CD20.

In one embodiment, the PEGylated anti-cancer agent comprises PEGylated liposomal doxorubicin or PEGylated liposomal vinorelbine. In a preferred embodiment, the PEGylated anti-cancer agent is PEGylated liposomal doxorubicin.

In another embodiment, the cancer comprises triple-negative breast cancer.

In one embodiment, the at least one monomeric bispecific PEG engager comprises a first monomeric bispecific PEG engager and a second monomeric bispecific PEG engager, wherein the target bound to the first monomeric bispecific PEG engager is different from the target bound to the second monomeric bispecific PEG engager.

In another embodiment, the heavy-chain for binding to PEG of the monomeric bispecific PEG engager comprises a CDR1 having the sequence of SEQ ID NO: 3, a CDR2 having the sequence of SEQ ID NO: 4 and a CDR3 having the sequence of SEQ ID NO: 5; and the light-chain for binding to PEG of the monomeric bispecific PEG engager comprises a CDR1 having the sequence of SEQ ID NO: 6, a CDR2 having the sequence of SEQ ID NO: 7 and a CDR3 having the sequence of SEQ ID NO: 8.

The present invention also provides a method for diagnosing a cell-mediated disorder in a subject, comprising: (1) administering to the subject at least one monomeric bispecific polyethylene glycol (PEG) engager that specifically binds to PEG and to targets on cells mediating the disorder; (2) administering to the subject a PEGylated diagnostic agent; and (3) detecting locations of the PEGylated diagnostic agent in the subject; wherein the PEGylated diagnostic agent is located in the cells upon binding to the at least one monomeric bispecific PEG engager bound to the cells, thereby diagnosing the cell-mediated disorder.

In one embodiment, the PEGylated diagnostic agent is administered after the clearance rate of the at least one monomeric bispecific PEG engager is higher than 90% in the subject.

In another embodiment, the targets comprise epidermal growth factor receptor (EGFR), insulin-like growth factor receptor, human epidermal growth factor receptor 2 (HER2), HER3, HER4, c-Met, CD19, CD20, CD5, CD21, CD25, CD37, CD30, CD33, CD45, CAMPATH-1, A33, G250, folate-binding protein, PSMA, GD2, GD3, GM2, Lewis Y, CA-125, CA19-9, IL2 receptor, tenascin, metalloproteinases or FAR In a preferred embodiment, the targets comprise EGFR, CD19 or CD20.

In one embodiment, the PEGylated diagnostic agent is a fluorescently or radioactively labeled PEGylated nanoparticle.

In one embodiment, the at least one monomeric bispecific PEG engager comprises a first monomeric bispecific PEG engager and a second monomeric bispecific PEG engager, wherein the target bound to the first monomeric bispecific PEG engager is different from the target bound to the second monomeric bispecific PEG engager.

In another embodiment, the heavy-chain for binding to PEG of the monomeric bispecific PEG engager comprises a CDR1 having the sequence of SEQ ID NO: 3, a CDR2 having the sequence of SEQ ID NO: 4 and a CDR3 having the sequence of SEQ ID NO: 5; and the light-chain for binding to PEG of the monomeric bispecific PEG engager comprises a CDR1 having the sequence of SEQ ID NO: 6, a CDR2 having the sequence of SEQ ID NO: 7 and a CDR3 having the sequence of SEQ ID NO: 8.

### EXAMPLES

Example 1: Expression and Purification of Bispecific PEG Engagers

Monovalent anti-PEG bispecific antibodies were generated by fusing the Fab fragment of a humanized antibody derived from anti-PEG antibody 6.3 (Kao et al. 2014, Biomaterials 35:9930-9940) to single chain antibodies with specificity for EGFR or CD19.

Anti-PEG Fab-based bispecific PEG engager antibodies were generated by cloning the mouse V_{L} and V_{H} domains of the 6.3 antibody from cDNA prepared from the 6.3 hybridoma (see Kao et al.). The anti-PEG antibody was humanized by first aligning the V_{H} and V_{L} sequences of the mouse 6.3 antibody to human immunoglobulin germline sequences using the IgBLAST program (found on the World Wide Web at ncbi.nlm.nih.gov/igblast/). The human germline V_{H} IGHV7-4-1*02 and V_{L} IGKV4-1*01 exons were selected based on the degree of framework homology. The complementarity-determining regions of mouse 6.3 V_{H} and V_{L} domains were then grafted onto human V_{H} IGHV7-4-1*02 and V_{L} IGKV4-1*01 genes using assembly PCR. Human immunoglobulin G1 (IgG₁) Ck and CH₁ constant domains were cloned from cDNA synthesized from extracted human peripheral blood mononuclear cell RNA. Humanized 6.3 V_{L}-Ck and 6.3 V_{H}-CH₁ domains were assembled by overlap polymerase chain reaction from humanized 6.3 V_{L} (SEQ ID NO: 2) and humanized 6.3 V_{H} (SEQ ID NO: 1) and human Ck and CH₁ fragments. To construct a pAS3w.Ppuro-PEG engager plasmid, the humanized 6.3 V_{L}-Ck and 6.3 V_{H}-CH₁ were joined by a composite internal ribosome entry site bicistronic expression peptide linker and inserted into the plasmid pAS3w.Ppuro obtained from National RNAi Core Facility, Institute of Molecular Biology/Genomic Research Center, Academia Sinica, Taiwan.

The hBU12 (anti-human CD19) and Necitumumab (IMC-11F8, anti-human EGFR) single chain dsFv were synthesized by assembly PCR based on the V_{H} and V_{L} sequences of hBU12 and Necitumumab from U.S. Pat. Nos. 7,968,687 and 7,598,350, respectively. The dsFv DNA fragments were digested with MfeI I and Mlu I and then subcloned into the pAS3w.Ppuro -PEG engager plasmid downstream of a GGGGS (SEQID NO: 9) peptide linker linked to the C terminus of the 6.3 Fab and upstream of a poly-histidine tag to generate pAS3w.Ppuro-PEG engager^{CD19} and pAS3w.Ppuro-PEG engager^{EGFR}.

293FT/PEG engager^{CD19} and 293FT/PEG engager^{EGFR} cells that stably secreted PEG engager^{CD19} and PEG engager^{EGFR} were generated by lentiviral transduction. Recombinant lentiviral particles were packaged by co-transfection of pAS3w. Ppuro-pAS3w.Ppuro-PEG engager^{CD19} and pAS3w.Ppuro-PEG engager^{EGFR} (7.5 μg) with packaging plasmid pCMVDR8.91 (6.75 μg) and VSV-G envelope plasmid pMD.G (0.75 μg) using 45 μl TransIT-LT1 transfection reagent (Minis Bio) in 293FT cells grown in a 10 cm culture dish to 90% confluency. After 48 h, lentiviral particles were collected and concentrated by ultracentrifugation (Beckman SW 41 Ti Ultracentrifuge Swinging Bucket Rotor, 50,000×g, 1.5 h, 4° C.).

Lentiviral particles were suspended in culture medium containing 5 μg/ml polybrene and filtered through a 0.45 μm filter. 293FT cells were seeded in six-well plates (1×10⁵ cells per well) 1 day before viral infection. Lentivirus-containing medium was added to the cells and then centrifuged for 1.5 h (500×g, 32° C.). The cells were selected in puromycin (5 μg/ml) to generate stable cell lines. 5×10⁷ 293FT/PEG engager^{CD19} or 293FT/PEG engager^{EGFR} cells in 15 ml DMEM culture medium were cultured in CELLine adhere 1000 bioreactors (INTEGRA Biosciences AG) and the medium was collected every 7 to 10 days.

Polyhistidine-tagged monovalent bispecific antibodies were purified on a Co²⁺-TALON column (GE Healthcare Life Sciences). Protein concentrations were determined by the bicinchoninic acid protein assay (Thermo Fisher Scientific).

Example 2: Characterization of Bispecific PEG Engagers

The PEG engager^{CD19} and PEG engager^{EGFR} have molecular weights of 78 kDa and 79 kDa, respectively, as determined by matrix-assisted laser desorption/ionization time-of-flight (MALDI-TOF) mass spectrometry. Size-exclusion high-performance liquid chromatography analysis showed a major peak corresponding to a monomer with minimal aggregation. The melting temperatures of PEG engager^{CD19} and PEG engager^{EGFR} were determined by differential scanning calorimetry. PEG engager^{CD19} and PEG engager^{EGFR} had melting temperatures, respectively, of 75° C. and 75.8° C., both of which are higher than the typical melting temperature of a Fab fragment, i.e., 61.9° C. to 69.4° C., indicating that both engagers had good thermal stability.

Equilibrium binding of the PEG engagers to PEG and to their specific ligand was analyzed by microscale thermophoresis as follows. HEPES buffered saline/CHAPS buffer (10 mM HEPES, 150 mM NaCl, 3 mM EDTA, 0.05% CHAPS, pH=7.4) was used for sample preparation. To determine the PEG-binding affinity of PEG engagers, 5 nM of Cy5-conjugated methoxy PEG5k (Nanocs) was mixed at a 1:1 volume ratio with graded concentrations (0.24-500 nM) of PEG engager^{CD19} or PEG engager^{EGFR} antibodies. To analyze the tumor antigen-binding affinity of PEG engagers, 2 nM of Alexa Fluor 647-conjugated PEG engager^{CD19} or PEG engager^{EGFR} were mixed at a 1:2 volume ratio with graded concentrations (0.027-180 nM) of recombinant CD19 or EGFR proteins (Sino Biological Inc.). The samples were incubated for 5 min. at room temperature and loaded into standard capillary tubes and heated at 5% LED and 40% laser power for 30 s, cooled for 10 s, and measured on a NanoTemper Monolith NT.115 instrument (NanoTemper Technologies GmbH). All experiments were performed in triplicate.

The results are shown in Table 1 below.

TABLE 1
Dissociation constants of bispecific PEG engagers

| engager | K_{D} for PEG | K_{D} for specific ligand |
|---|---|---|
| PEG engager^{EGFR} | 7.55 ± 1.13 nM | 0.96 ± 0.23 nM |
| PEG engager^{CD19} | 7.6 ± 1.05 nM | 3.6 ± 0.35 nM |

Example 3: Specific Delivery of PEGylated Nanoparticles by PEG Engagers

Cancer cell lines expressing different levels of EGFR were tested to determine whether PEG engager^{EGFR} could specifically deliver PEGylated nanoparticles into EGFR-expressing (EGFR⁺) cancer cells. In particular, cancer cell-specific uptake of PEGylated nanoparticles mediated by PEG engager^{EGFR} was examined in EGFR⁺ and non-expressing (EGFR⁻) breast cancer cells by real-time confocal microscopy as set forth, infra.

Coverslips (30 mm) in cell cultivation POCmini chambers (perfusion, open and closed; PeCon GmbH) were coated with 10 μg/ml poly-L-lysine (Sigma-Aldrich) in PBS for 30 min. at room temperature. The coverslips were washed twice with PBS and then 5×10⁴ MDA-MB-468 (EGFR⁺), A431 (EGFR⁺), and MCF7 (EGFR⁻) cancer cells were each seeded on separate coverslips. Cancer cell-specific uptake of PEGylated nanoparticles was examined by staining the cells with 10 μg/ml of PEG engager^{CD19} or PEG engager^{EGFR} antibodies at 37° C. for 30 min. in medium containing 1 μg/ml of Hoechst 33342 (Thermo Fisher Scientific). The cells were washed twice with PBS to remove unbound PEG engagers, and incubated with 8 nM PEGylated Qtracker 655 non-targeted quantum dots (PEG-Qdot655; Thermo Fisher Scientific) in medium (RPMI-1640, 10% FBS). Cells were visualized by real-time imaging on an Axiovert 200M Confocal Microscope (Carl Ziess Inc.) at excitation and emission wavelengths of 350 nm and 461 nm for Hoechst 33342 and 350 nm and 675 nm for PEG-Qdot655 at 37° C., 5% CO₂.

Both MDA-MB-468 triple negative breast cancer (TNBC) and A431 non-TNBC cells express EGFR but not CD19. MCF7 non-TNBC cells express neither EGFR nor CD19. PEG engager^{EGFR} mediated rapid accumulation of PEG-Qdot655 in both MDA-MB-468 and A431 cells, but not in MCF7 cells. By contrast, no uptake of PEG-Qdot655 was observed in MDA-MB -468, A431, and MCF7 cells treated with control PEG engager^{CD19}. Clearly, PEG engager^{EGFR} can deliver PEGylated nanoparticles into breast cancer cells that express EGFR.

Example 4: Conditional Internalization of PEGylated Nanoparticles

The ability of PEG engager^{EGFR} to trigger PEGylated nanoparticle-dependent receptor-mediated internalization was examined by confocal microscopy.

Five milligrams of purified PEG engager^{CD19} or PEG engager^{EGFR} antibodies in coupling buffer (0.1 M sodium bicarbonate, pH=8.0) was mixed with a 10-fold molar excess of Alexa Fluor 647 succinimidyl ester (Thermo Fisher Scientific) in dimethyl sulfoxide for 2 h at room temperature to produce Alexa Fluor 647-conjugated PEG engager^{CD19} and PEG engager^{EGFR}. One-tenth volume of 1 M glycine (pH=8.0) was added to stop the reaction. The labeled PEG engagers were dialysed (molecular weight cutoff 12,000-14,000 daltons) against PBS to remove free Alexa Fluor 647, sterile filtered, and stored at −80° C.

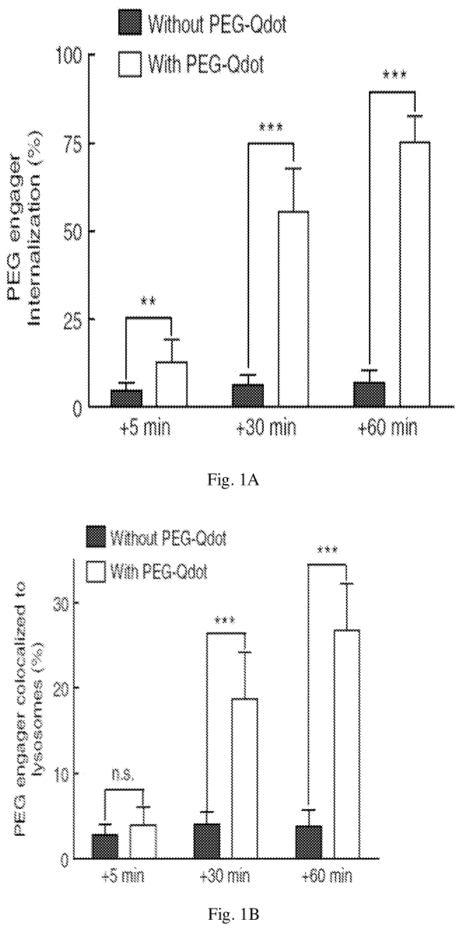

Conditional internalization of PEGylated nanoparticles was determined by incubating MDA-MB-468 or BT-20 cells with 10 μg/ml of Alexa Fluor 647-conjugated PEG engager^{EGFR} (excitation/emission, 650 nm/675 nm) at 37° C. for 30 min. in medium containing 1 μg/ml of Hoechst 33342 and 100 nM of LysoTracker Red DND-99, a lysosome stain. After washing, the cells were incubated at 37° C. for 1 h or 9 h and imaged using the Axiovert 200M confocal microscope, followed by real-time cell imaging after adding 8 nM of PEG-Qdot655 solution. The percentages of internalized PEG engagers and PEG-Qdots were calculated by dividing the fluorescence of the intracellular regions by the whole-cell fluorescence based on the bright field cell images using ZEN 2011 software (blue edition; Carl Zeiss, Jena, Germany). The results are shown in FIGS. 1A and 1B.

The data showed that the PEG engager^{EGFR} remained on the plasma membrane of MDA-MB-468 cells at 37° C. for 1 h with almost no internalization. See FIG. 1A. PEG engager^{EGFR} displayed limited endocytosis, i.e., internalization, in MDA-MB-468 cells even after 9 h. Upon addition of PEG-Qdot655 to the cells, PEG engager^{EGFR} on the cell membrane together with the PEG-Qdot655were rapidly internalized into the cells. Co-localization of PEG engager^{EGFR} with PEG-Qdot655 or with lysosomes verified that PEG engager^{EGFR} can conditionally stimulate endocytosis of PEGylated nanoparticles and then localize in lysosomes. See FIG. 1B.

Example 5: Efficacy of PEG Engager-Directed Liposomal Drugs in Vitro.

PEG engager^{EGFR} was tested for its ability to enhance the in vitro anti-proliferative activity of a drug-loaded nanocarrier, e.g., liposome, in the following different types of cancer cells that express either wild-type EGFR or mutated EGFR. MDA-MB-231, MDA-MB-468, and BT-20 are TNBC cancer cell lines that express wild-type EGFR; SKBR3 is a non-TNBC breast adenocarcinoma cell line that expresses wild-type EGFR; and PC9 is non-small cell lung cancer cell line that expresses EGFR with a delta E746-A750 deletion in the tyrosine kinase domain.

PEGylated liposomal drug-loaded nanocarriers were produced as follows. Distearoyl phosphatidylcholine, 1,2-distearoylsn-glycero-3-phosphoethanolamine-N-[methoxy(polyethylene glycol)-2000 (DSPE-PEG2000) and cholesterol (Avanti Polar Lipids, Inc.) were dissolved in chloroform at a 65:5:30 molar ratio, respectively. A dried lipid film was formed at 65° C. by rotary evaporation (Buchi, Rotavapor RII) and rehydrated in Tris-buffered saline (TBS; 50 mM Tris-HCl, 150 mM NaCl, pH 7.4) at 65° C. to a final lipid concentration of 20 mg/ml. This liposomal suspension was subjected to 10 freeze/thaw cycles in liquid nitrogen and a heated water bath at 80° C., followed by 21 extrusions at 75° C. through 400, 200, and 100 nm polycarbonate membranes using a mini-extruder (Avanti Polar Lipids, Inc.). The final lipid concentration was measured by Bartlett's assay and adjusted to 13.9 μmol/ml with TBS before use.

Similar PEGylated liposomes loaded with doxorubicin, i.e., doxisomes, or loaded with vinorelbine were also obtained. Free drug was used as a positive control.

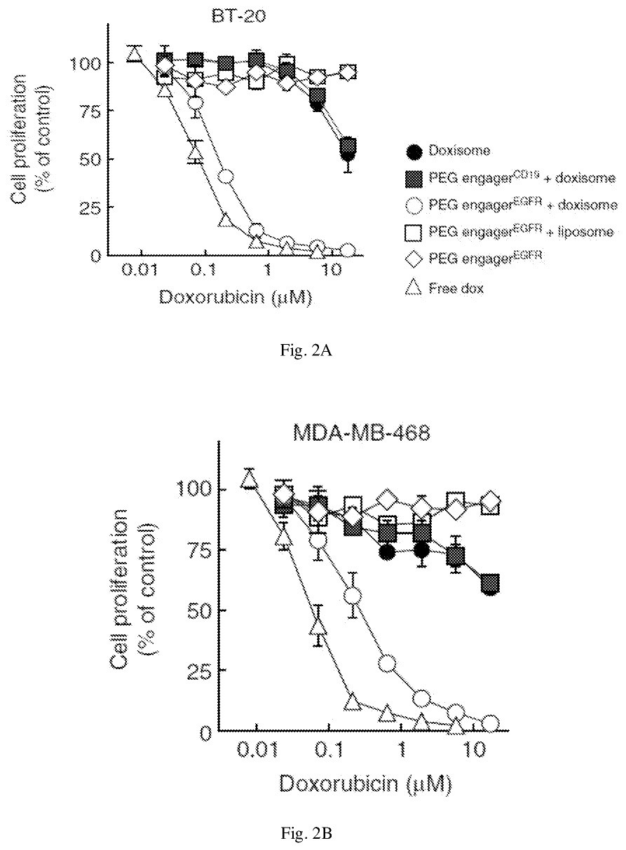

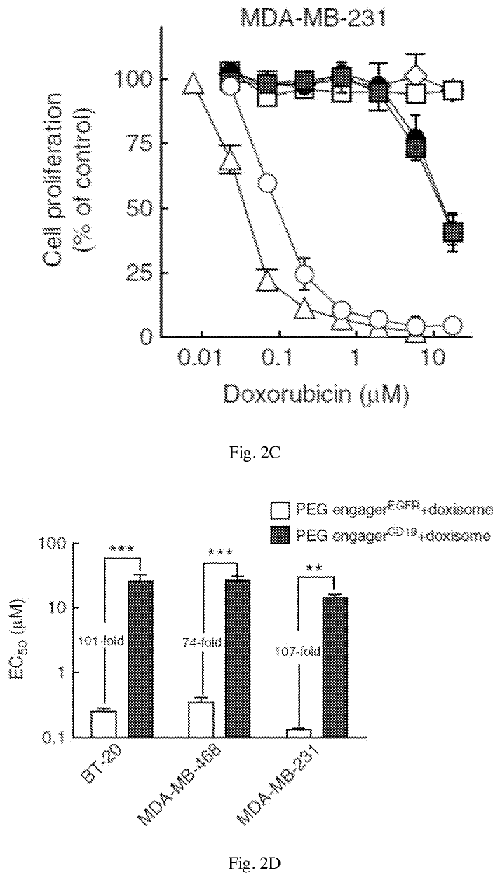

The above cell lines, as well as PC3, SKBR3, SK-MES-1, Hut125, Caski, HT29, H2170, LS174T, HepG2, and SW480 cells were seeded in 96-well plates at 10,000 cells per well and cultured overnight. Serial dilutions of free doxorubicin or vinorelbine were directly added to the cells as positive controls. Fifteen microgram per ml of PEG engager^{CD19} or PEG engager^{EGFR} were added to the cells followed after 30 min. at 37° C. by addition of (i) graded concentrations of PEGylated liposomal doxorubicin (Doxisome, 13.9 μmol/ml lipid concentration, Taiwan Liposome Company Ltd., Taipei, Taiwan), (ii) PEG-liposomal vinorelbine (provided by Dr Han-Chung Wu, Research Fellow, Institute of Cellular and Organismic Biology, Academia Sinica, Taiwan), or (iii) empty liposomes to triplicate wells and incubation at 37° C. for 4 h. The cells were subsequently washed once and incubated for an additional 72 h in fresh culture medium and then pulsed for 18 h with ³H-thymidine (1 μCi per well). The percent inhibition of ³H-thymidine incorporation into cellular DNA was calculated. The results are depicted in FIGS. 2A-2D.

The results showed that PEG engager^{EGFR} significantly enhanced the anti-proliferative activity of Doxisomes against EGFR⁺ cancer cells, as compared to drug-loaded liposomes alone, drug-loaded liposomes plus PEG engager^{CD19}, or empty liposomes with PEG engager^{EGFR}. See FIGS. 2A-C. A similar result was obtained with PEG-liposomal vinorelbine. The half maximal effective concentration (EC₅₀) values for PEG engager^{EGFR} targeted Doxisome in BT-20, MDA-MB-468, and MDA-MB-231 cells were 101-fold, 74-fold, and 107-fold lower than that of control PEG engager^{CD19} targeted Doxisome, respectively. See FIG. 2D. Neither PEG engager^{EGFR} nor PEG engager^{CD19} altered the sensitivity of HepG2 cells (EGFR⁻) to Doxisomes. In contrast to wild-type BT-20 cells, PEG engager^{EGFR} did not enhance the anti-proliferation activity of Doxisome in BT-20/shEGFR cancer cells (BT-20 cells treated with short hairpin RNA to knockdown the expression of EGFR) as compared with drug-loaded liposome alone or drug-loaded liposome plus PEG engager^{CD19}. In sum, PEG engager^{EGFR} significantly increases the anticancer activity of PEGylated medicines, i.e., Doxisome and PEG-liposomal vinorelbine, against EGFR⁺ cancer cells.

Example 6: Pre-Existing Anti-PEG Antibodies Do Not Effectively Compete with PEG Engager^{EGFR}.

Pre-existing anti-PEG antibodies in healthy donors might negatively impact PEG engager targeting of PEGylated medicines by blocking engager binding to PEG on the nanomedicines. Anti-PEG antibody concentrations in healthy human plasma samples were measured using an anti-PEG enzyme-linked immunosorbent assay (ELISA). Pre-existing anti-PEG IgG concentrations ranged from 0.3 μg/ml to 237.5 μg/ml with a mean concentration of 5.75±16.0 μg/ml in 386 anti-PEG positive samples. A human serum sample containing 51.4 mg/ml anti-PEG IgG was tested to determine whether it altered the in vitro anti-proliferative activity of liposomal anti-cancer drugs. The results showed that PEG engager^{EGFR} plus Doxisome tested in the presence of 20% of anti-PEG IgG-positive human serum or 20% control human serum had similar EC₅₀ values for inhibiting the proliferation of MDA-MB-468 cells. These results show that pre-existing anti-PEG antibodies in patients do not effectively compete with PEG engager^{EGFR}. Not to be bound by theory, this may be due to a higher anti-PEG affinity of the PEG engager and abundant PEG chains on the Doxisomes.

Example 7: Pharmacokinetics and Tumor Targeting of the PEG Engager.

Pre-targeting of PEG engagers to tumors may allow for subsequent accumulation and endocytosis of PEGylated nanocarriers into cancer cells. The in vivo pharmacokinetics of PEG engagers was examined to determine a reasonable time point for administration of PEGylated nanocarriers after administration of a PEG engager.

NSG mice were intravenously injected with 150 μg PEG engager^{CD19} or PEG engager^{EGFR} and blood samples were periodically collected from the tail vein of the mice. Plasma was prepared by centrifugation for 5 min. at 12,000×g. PEG engager levels in plasma were determined by quantitative sandwich ELISA as follows. Maxisorp 96-well microplates were coated with 50 μl per well 2 mg/ml of anti-6 His tag antibody (GeneTex) in bicarbonate buffer, pH 8.0 for 4 h at 37° C. and then incubated at 4° C. overnight. The plates were blocked with 200 μl per well 5% skim milk in PBS for 2 h at room temperature and then washed with PBS three times. Graded concentrations of PEG engager^{CD19}, PEG engager^{EGFR}, or plasma samples in dilution buffer (2% skim milk in PBS) were added to the wells for 2 h at room temperature. After washing with PBS four times, the plates were stained with 50 μl per well horseradish peroxidase-conjugated anti-human IgG Fab antibody (Jackson ImmunoResearch Laboratories) at 5 μg/ml. The plates were washed 6 times with PBS before adding 100 μl per well ABTS solution (0.4 mg/ml 2,20-azino-di(3-ethylbenzthiazoline-6-sulfonic acid), 0.003% H₂O₂, 100 mM phosphate citrate, pH 4.0 and incubating for 30 min. at room temperature. The absorbance of the wells at 405 nm was measured on a microplate reader. The initial and terminal half-lives of the PEG engagers were estimated by fitting the data to a two-phase exponential decay model using Prism 5 software (Graphpad Software).

The half-lives of PEG engager^{EGFR} and PEG engager^{CD19} were approximately 2.1 h and 2.2 h, respectively. Almost 90% of both PEG engagers were cleared from the circulation by 5 h after injection.

The uptake and retention of PEGylated compounds in mice bearing established high EGFR-expressing tumors (MDA-MB-468 and A431) or low EGFR-expressing tumors (HepG2) was examined in vivo after treatment with PEG engagers as set forth below.

A PEGylated near-infrared probe was prepared by dissolving 4arm-PEG10K-NH₂ (Laysan Bio) in dimethyl sulfoxide at 2 mg/ml and mixing it with a six-fold molar excess of NIR-797 isothiocyanate (Santa Cruz Biotechnology) in dimethyl sulfoxide for 2 h at room temperature to produce a 4arm-PEG10K- NIR-797 probe. The probe was diluted in a fivefold volume of ddH₂O and dialysed (molecular weight cutoff ˜12,000-14,000 daltons) against ddH₂O to remove free NIR-797 isothiocyanate. The probes were sterile filtered and stored at 80° C.

BALB/c nude or NOD SCID mice bearing 100 mm³ subcutaneous MDA-MB-468, A431, or HepG2 xenograft tumors were each intravenously injected with 6 mg/kg PEG engager^{CD19} or PEG engager^{EGFR}. Five hours after PEG engager injection, the mice were intravenously administrated with the 4arm-PEG10KNIR-797 probe at 5 mg/kg. Pentobarbital anesthetized mice were imaged with an IVIS Spectrum imaging system (excitation, 745 nm; emission, 840 nm; PerkineElmer) at 24, 48, and 72 h after injection.

The in vivo imaging results showed that the fluorescence signal in PEG engager^{EGFR} targeted tumors was significantly enhanced as compared to the PEG engager^{CD19} control group. In an exemplary experiment in MDA-MB-468 tumor-bearing mice, the fluorescence intensity in PEG engager^{EGFR} targeted tumors at 24, 48 and 72 h was, respectively, 2.7-fold, 2.1-fold, and 2.8-fold greater, as compared to control PEG engager treated tumors at these time points. Neither PEG engager^{EGFR} nor PEG engager^{CD19} significantly enhanced the fluorescence signal in HepG2 (low EGFR expression levels) tumor-bearing mice.

Example 8: Anti-Tumor Activity of Pre-Targeted PEG Engager.

Anti-tumor activity of PEG engager was tested in mice bearing human MDA-MB-468 TNBC or MDA-MB-231 TNBC xenografts following the procedure below.

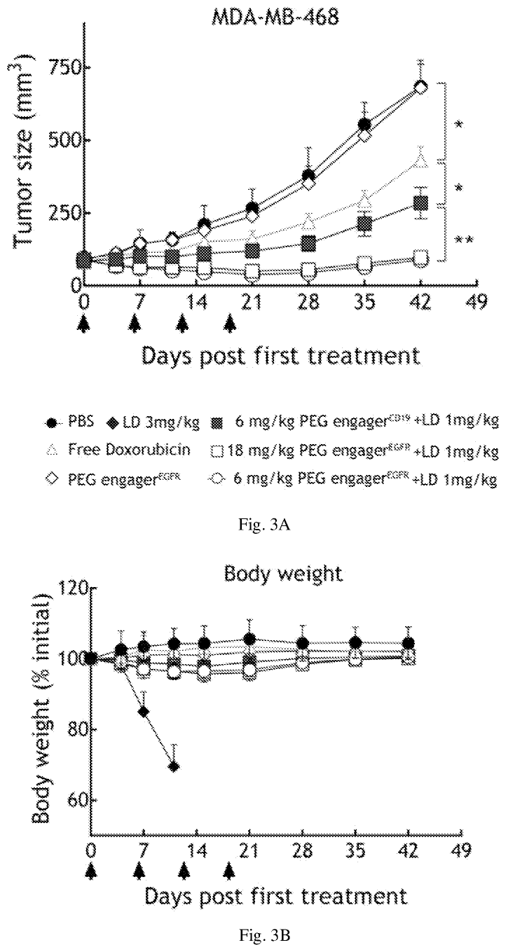

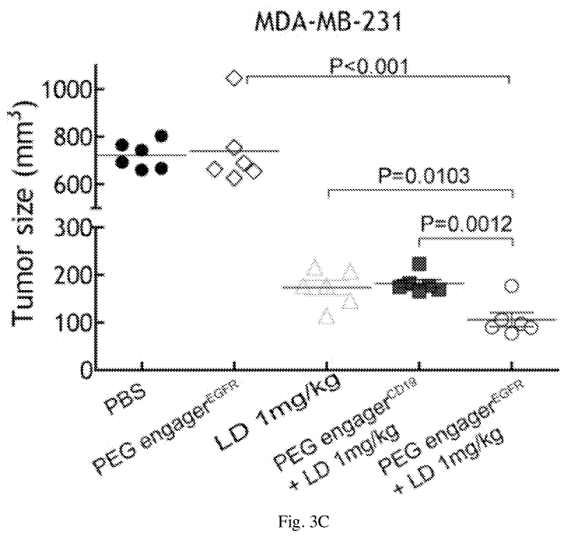

Groups of severe combined immunodeficiency (SCID) mice bearing 44.7±10.7 mm³ MDA-MB-231 (n=6) or 84.3±4.3 mm³ subcutaneous MDA-MB-468 (n=8) tumors on their right flanks were intravenously injected with PBS or with 6 or 18 mg/kg PEG engagers. After 5 h, the mice were intravenously administrated with free doxorubicin (3 mg/kg) or Doxisome (1 mg/kg or 3 mg/kg). This treatment was repeated once a week for a total of 4 weeks. Tumour sizes were measured every 7 days. The results are shown in FIG. 3A-C.

Mice treated with PEG engager^{EGFR} alone displayed tumor growth similar to that shown in mice treated with PBS. As expected, free doxorubicin suppressed tumor growth as compared to treatment of mice with PBS. PEG engager^{CD19} combined with 1 mg/kg Doxisome® or 1 mg/kg Doxisome® alone displayed similar and better suppression of tumor growth as compared to treatment of mice with free doxorubicin or with PBS vehicle. PEG engager^{EGFR} plus Doxisome® significantly suppressed TNBC tumor growth, as compared to mice treated with Doxisome® alone. See FIGS. 3A and 3C.

The maximum-tolerated dose of doxorubicin in SCID mice is typically 2.5-3 mg/kg due to defective DNA repair in these mice. See Haun et al. 2010, Nat. Nanotechnol. 5:660-665. Increasing the dose of Doxisome® to 3 mg/kg did not provide better therapeutic activity because the mice experienced significant body weight loss and early death. See FIG. 3B.

The above results demonstrate that pre-targeting PEG engager^{EGFR} to EGFR over-expressing TNBC tumors can markedly enhance the therapeutic efficacy of PEGylated liposomal doxorubicin with limited side effects as estimated by body weight loss analysis.

Example 9: Off-Target Effects of PEG Engager Mediated Therapy

The density of EGFRs on cells might be an important factor for conditional internalization of PEGylated nanocarriers by PEG engager^{EGFR}. The levels of EGFR expression on cancer cell lines was measured and compared to the EC₅₀ value of PEG engager^{EGFR} plus Doxisome® on cancer cell proliferation in vitro.

Surface expression of EGFR in human hepatocytes, PC3, SKBR3, SK-MES-1, Hut125, Caski, HT29, H2170, LS174T, HepG2, SW480, MDA-MB-231, MDA-MB-468, and BT-20 cells was determined by staining the cells with monoclonal mouse IgG anti-human EGFR (Santa Cruz Biotechnology) at 5 μg/ml in staining buffer (PBS containing 0.1% bovine serum albumin) for 30 min. at 4° C. Binding of the anti-EGFR antibody was detected by incubating with 5 μg/ml Alexa Fluor 647-conjugated goat Ig anti-mouse IgG antibody (Thermo Fisher Scientific,), followed by washing twice with cold PBS to remove unbound antibodies. The surface fluorescence of 10⁴ viable cells was measured using a FACScaliber flow cytometer (Becton Dickinson) and analyzed with Flowjo software (Tree Star Inc.).

EC₅₀ values for doxisome/PEG engager^{EGFR} treatment were determined as set forth in Example 5, supra.

The data showed a linear correlation between the logarithm of EGFR expression levels on cancer cell lines and the logarithm of the anti-proliferative EC₅₀ values of doxisome/PEG engager^{EGFR} treatment (R²=0.702).

It has been reported that an off-target effect of EGFR-targeted therapies is hepatotoxicity. See Hapuarachchige et al. 2016, Sci. Rep. 6, 24298. Yet, measuring EGFR expression in normal human hepatocytes as described above is relatively low (mean fluorescence intensity=38), suggesting that PEG engager^{EGFR} therapy will have low off-target toxicity.

Example 10: PEG Engager^{EGFR} Can Inhibit EGFR Signaling

To investigate whether PEG engager^{EGFR} can inhibit EGFR signaling, EGFR-positive A431 cells were untreated or stimulated with 5 nM EGF and then co-incubated with PEG engagers or control antibodies. Phosphorylation of EGFR and Erk were detected by western blotting using anti-phospho EGFR or anti-phospho ERK antibodies. Total EGFR and tubulin served as loading controls.

As expected, negative control antibody Herceptin (anti-HER2 IgG) and PEG engager^{CD19}, both at 50 nM, did not inhibit EGF-induced phosphorylation of EGFR and Erk. By contrast, 50 nM of Erbitux (monoclonal anti-EGFR IgG) and 50 nM of PEG engager^{EGFR} inhibited phosphorylation of the EGFR and Erk in EGF-treated cells.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method for treating cancer, comprising:
(1) identifying a subject suffering from cancer;
(2) administering to the subject at least one monomeric bispecific polyethylene glycol (PEG) engager that specifically binds to PEG and to targets on cancer cells in the subject; and
(3) administering to the subject a PEGylated anti-cancer agent after receiving the at least one monomeric bispecific PEG engager,
wherein the PEGylated anti-cancer agent is internalized into the cancer cells upon binding to the monomeric bispecific PEG engager bound to the cancer cells, thereby killing the cancer cells.

2. The method of claim 1, wherein the PEGylated anti-cancer agent is administered after the clearance rate of the at least one monomeric bispecific PEG engager is higher than 90% in the subject.

3. The method of claim 1, wherein the targets comprise epidermal growth factor receptor (EGFR), insulin-like growth factor receptor, human epidermal growth factor receptor 2 (HER2), HER3, HER4, c-Met, CD19, CD20, CD5, CD21, CD25, CD37, CD30, CD33, CD45, CAMPATH-1, A33, G250, folate-binding protein, PSMA, GD2, GD3, GM2, Lewis Y, CA-125, CA19-9, IL2 receptor, tenascin, metalloproteinases or FAP.

4. The method of claim 1, wherein the targets comprise EGFR, CD19 or CD20.

5. The method of claim 1, wherein the PEGylated anti-cancer agent comprises PEGylated liposomal doxorubicin or PEGylated liposomal vinorelbine.

6. The method of claim 1, wherein the PEGylated anti-cancer agent is PEGylated liposomal doxorubicin.

7. The method of claim 1, wherein the cancer comprises triple-negative breast cancer.

8. The method of claim 1, wherein the at least one monomeric bispecific PEG engager comprises a first monomeric bispecific PEG engager and a second monomeric bispecific PEG engager, wherein the target bound to the first monomeric bispecific PEG engager is different from the target bound to the second monomeric bispecific PEG engager.

9. The method of claim 1, wherein the heavy-chain for binding to PEG of the monomeric bispecific PEG engager comprises a CDR1 having the sequence of SEQ ID NO: 3, a CDR2 having the sequence of SEQ ID NO: 4 and a CDR3 having the sequence of SEQ ID NO: 5; and the light-chain for binding to PEG of the monomeric bispecific PEG engager comprises a CDR1 having the sequence of SEQ ID NO: 6, a CDR2 having the sequence of SEQ ID NO: 7 and a CDR3 having the sequence of SEQ ID NO: 8.

10. A method for diagnosing a cell-mediated disorder in a subject, comprising:
(1) administering to the subject at least one monomeric bispecific polyethylene glycol (PEG) engager that specifically binds to PEG and to targets on cells mediating the disorder;
(2) administering to the subject a PEGylated diagnostic agent; and
(3) detecting locations of the PEGylated diagnostic agent in the subject;
wherein the PEGylated diagnostic agent is located in the cells upon binding to the at least one monomeric bispecific PEG engager bound to the cells, thereby diagnosing the cell-mediated disorder.

11. The method of claim 10, wherein the PEGylated diagnostic agent is administered after the clearance rate of the at least one monomeric bispecific PEG engager is higher than 90% in the subject.

12. The method of claim 10, wherein the targets comprise epidermal growth factor receptor (EGFR), insulin-like growth factor receptor, human epidermal growth factor receptor 2 (HER2), HER3, HER4, c-Met, CD19, CD20, CD5, CD21, CD25, CD37, CD30, CD33, CD45, CAMPATH-1, A33, G250, folate-binding protein, PSMA, GD2, GD3, GM2, Lewis Y, CA-125, CA19-9, IL2 receptor, tenascin, metalloproteinases or FAP.

13. The method of claim 10, wherein the targets comprise EGFR, CD19 or CD20.

14. The method of claim 10, wherein the PEGylated diagnostic agent is a fluorescently or radioactively labeled PEGylated nanoparticle.

15. The method of claim 10, wherein the at least one monomeric bispecific PEG engager comprises a first monomeric bispecific PEG engager and a second monomeric bispecific PEG engager, wherein the target bound to the first monomeric bispecific PEG engager is different from the target bound to the second monomeric bispecific PEG engager.

16. The method of claim 10, wherein the heavy-chain for binding to PEG of the monomeric bispecific PEG engager comprises a CDR1 having the sequence of SEQ ID NO: 3, a CDR2 having the sequence of SEQ ID NO: 4 and a CDR3 having the sequence of SEQ ID NO: 5; and the light-chain for binding to PEG of the monomeric bispecific PEG engager comprises a CDR1 having the sequence of SEQ ID NO: 6, a CDR2 having the sequence of SEQ ID NO: 7 and a CDR3 having the sequence of SEQ ID NO: 8.


SEQUENCE LISTING (9 sequences)

SEQ ID NO: 1 - protein, 114 aa, Artificial Sequence
  humanized VH chain
QVQLVQSGSELKKPGASVKVSCKASGYTFKNYGMNWVRQAPGQGLEWMGWINTYTGQPIYANDFKGRFVFSLDTSVSTAYLQISSLKAEDTAVYYCARDWGPYWGQGTLVTVSS

SEQ ID NO: 2 - protein, 112 aa, Artificial Sequence
  humanized VL chain
DIVMTQSPDSLAVSLGERATINCKSSQSVLYSSNQMNYLAWYQQKPGQPPKLLIYWASTRESGVPDRFSGSGSGTDFTLTISSLQAEDVAVYYCLQYLSSWTFGGGTKLEIK

SEQ ID NO: 3 - protein, 8 aa, Artificial Sequence
  H-CDR1
GYTFKNYG

SEQ ID NO: 4 - protein, 8 aa, Artificial Sequence
  H-CDR2
INTYTGQP

SEQ ID NO: 5 - protein, 7 aa, Artificial Sequence
  H-CDR3
ARDWGPY

SEQ ID NO: 6 - protein, 12 aa, Artificial Sequence
  L-CDR1
QSVLYSSNQMNY

SEQ ID NO: 7 - protein, 7 aa, Artificial Sequence
  L-CDR2
WASTRES

SEQ ID NO: 8 - protein, 8 aa, Artificial Sequence
  L-CDR3
LQYLSSWT

SEQ ID NO: 9 - protein, 5 aa, Artificial Sequence
  Synthetic linker
GGGGS